(19) United States
(12) Patent Application Publication
(10) Pub. No.: US 2025/0186186 A1
(43) Pub. Date: Jun. 12, 2025

(54) SYSTEM AND METHOD FOR DRY-PACKAGED HEART VALVE

(71) Applicant: EDWARDS LIFESCIENCES CORPORATION, Irvine, CA (US)

(72) Inventors: Vipul P. Rajpara, Lake Forest, CA (US); Martha Getaneh, Mission Viejo, CA (US); Jason Seng-Che Lam, Lake Forest, CA (US); Timothy William Hamilton, Orange, CA (US); Deidania Karina Paredes Feliz, Lake Forest, CA (US); Tara Christine Ziegenmeyer, Costa Mesa, CA (US); Sameer Dinmanishanker Upadhyaya, Irvine, CA (US)

(21) Appl. No.: 19/061,214

(22) Filed: Feb. 24, 2025

Related U.S. Application Data
(63) Continuation of application No. PCT/US2023/032264, filed on Sep. 8, 2023.
(60) Provisional application No. 63/375,216, filed on Sep. 9, 2022.

(51) Int. Cl. A61F 2/00; A61F 2/24

(52) U.S. Cl. CPC A61F 2/0095; A61F 2/2418

(57) ABSTRACT

A system for dry storage of a bioprosthetic heart valve comprising a valve housing and a storage tray. The valve housing has a cavity that is dimensioned to retain the heart valve and permits the addition of a hydrating liquid prior to implantation. A lid is pivotably movable between open position and a closed position to enclose the heart valve within the valve housing. The valve housing cavity is dimensioned to inhibit rotational and lateral movement of the bioprosthetic heart valve. The valve housing nests within a storage tray and mating surfaces position the valve housing in a specific orientation and help resist rotational movement of the valve housing relative to the storage tray. The storage tray has a gas-permeable membrane and the valve housing features apertures to permit passage of a sterilizing gas.

### CROSS-REFERENCE TO RELATED APPLICATIONS

The present application is a continuation of PCT patent application no. PCT/US2023/032264, filed on Sep. 8, 2023, which application claims priority to U.S. Provisional Patent Application No. 63/375,216, filed on Sep. 9, 2022, the disclosure of each of which is incorporated by reference herein in its entirety, including any drawings.

### TECHNICAL FIELD

This disclosure relates to a packaging for medical devices and, more particularly, to a method and system for dry-packaging a bioprosthetic valve without a liquid sterilant solution.

### BACKGROUND

The packaging of bioprosthetic heart valves have presented with numerous challenges. Bioprosthetic heart valves are conventionally packaged in jars filled with preserving solution for shipping and storage prior to use in the operating theater. To minimize the possibility of damage to the relatively delicate bioprosthetic heart valves, they are stabilized with bracketing structure to prevent contact with the inside of the jar.

Prior to implantation in a patient, the valve is removed from the jar and then rinsed with a stream of liquid, or immersed and agitated in a bath. Surgical prosthetic valves typically have a valve holder centrally located and sutured thereto, and the holders used for both are attached to the proximal end of the valve—to the inflow sewing ring for a mitral valve and to the outflow commissure tips for an aortic valve—so that an attached surgical delivery handle attached thereto extends proximally away from the implant site.

Glutaraldehyde is widely used as a storage solution due to its sterilant properties, but can contribute to calcification. Certain strategies to reduce glutaraldehyde content in the final product have been demonstrated to mitigate in vivo calcification.

One such strategy dehydrates the bioprosthetic tissue in a glycerol/ethanol mixture, sterilizes with ethylene oxide, and packages the final product “dry.” This process circumvents the potential toxicity and calcification effects of glutaraldehyde as a sterilant and storage solution. Because the dehydrated prosthetic heart valve can absorb ambient moisture, a moisture-barrier package system can be used.

There have been several methods proposed to use glycerin, alcohols, polyols, sugars, sugar alcohols, hydrophilic polymers, and combinations thereof as post-glutaraldehyde processing agents so that the resulting tissue is in a dehydrated or “dry” state rather than a wet state that is stored in excess glutaraldehyde. These approaches avoid the use of aqueous liquid aldehyde, or liquid sterilant as storage solutions for tissue and devices. Glycerol-based methods can be used in such storage systems, such as described in Parker et al. (Thorax 1978 33:638). Also, U.S. Pat. No. 6,534,004 (Chen et al.) describes the treatment of bioprosthetic tissue using polyhydric alcohols such as glycerol.

In view of the development of dehydrated-tissue heart valves, opportunities for alternative packaging for such valves arise that will provide combinations of a moisture barrier, reduced cost, and simplified field deployment.

### SUMMARY

This disclosure relates to a packaging for medical devices and, more particularly, to a method and system for dry packaging a bioprosthetic heart valve.

In one example, a system for dry storage of a bioprosthetic heart valve includes a valve housing that is dimensioned to removably retain a bioprosthetic heart valve. The bioprosthetic heart valve can comprise a stent having a stent diameter (dₛ) and a compressible fabric covering a portion of an external surface of the stent, the covered portion having a diameter (dₕᵥ) that is greater than the stent diameter (dₛ). The valve housing can comprise a cavity having an upper cavity opening, a cavity lip surrounding the upper cavity opening, a bottom wall, and an inner side wall. The cavity can be dimensioned to accommodate the bioprosthetic heart valve and to retain liquid for hydrating the bioprosthetic heart valve prior to implantation in a patient.

At least a portion of the opposing surfaces of the inner side wall can be configured to contact the bioprosthetic heart valve when the bioprosthetic heart valve is positioned within the cavity. Rotational and lateral movement of the bioprosthetic heart valve within the cavity can be inhibited by the contact between the opposing surfaces and the bioprosthetic heart valve. In one aspect, the opposing surfaces of the inner side wall can be spaced to compress only the compressible fabric of the bioprosthetic heart valve and not the stent. In accordance with this aspect, a diameter of the stent (dₛ) can remain substantially unchanged.

A lid that can be provided to enclose the upper cavity opening. The lid can be pivotably movable between an open position and a closed position. In the open position, the lid is pivoted away from the upper cavity opening to provide access to the cavity. In the closed position, the lid can be frictionally secured to a cavity lip surrounding the upper cavity opening. In one example, the lid can be pivotably movable between an open position exposing the upper cavity opening and a closed position to cover the upper cavity opening.

In the closed position, a lid inner surface can face the bottom wall and can be spaced at a distance (l_{lb}) that can be substantially the same as a length (lₕᵥ) of the bioprosthetic heart valve to inhibit movement of the bioprosthetic heart valve along a longitudinal axis of the bioprosthetic heart valve within the valve housing. In accordance with one aspect, the distance between the lid inner surface and the bottom wall (l_{lb}) is the same, if not slightly larger, than the length (lₕᵥ) of the bioprosthetic heart valve when the lid is in the closed position.

The lid can further comprise a plurality of undulations about its periphery. In the closed position, the plurality of undulations can permit the passage of a sterilizing gas into the cavity. In accordance with one aspect, a plurality of undulations can be formed from one or both of the lid periphery and the cavity lid. Thus, when the lid is in the closed position, at least a portion of the lid periphery and at least a portion of the cavity lip surrounding the upper cavity can be joined together such that the plurality of undulations can form apertures into the cavity.

In accordance with one aspect, the valve housing can further comprise an outer side wall that is spaced away from the inner side wall. The outer side wall can provide an enlarged cavity volume to retain liquid for hydrating the bioprosthetic heart valve.

In an example, the system can further comprise a storage tray within which the valve cavity can nest. The storage tray can comprise an upper tray opening and a microbial barrier membrane. The storage tray can be configured to receive and enclose the valve housing through the upper tray opening and can enclose the valve housing when the microbial barrier membrane is secured to a tray lip surrounding the upper tray opening.

In accordance with one aspect, the storage tray can be formed from a single, unitary piece of material.

In accordance with another aspect, the storage tray does not comprise separate parts that are physically joined together.

In accordance with another aspect, the valve housing can nest entirely within the storage tray and microbial barrier membrane.

In accordance with another aspect, the microbial barrier membrane can be permeable to a sterilizing gas.

In accordance with another aspect, the storage tray and the valve housing can be shaped to inhibit rotational movement of the valve housing relative to the storage tray when the valve housing enclosed within the storage tray.

In accordance with another aspect, the storage tray and the valve housing can each comprise mating surfaces configured to contact one another when the valve housing is placed within the storage tray. The mating surfaces can be shaped or configured to inhibit rotational and lateral movement of the valve housing within the storage tray.

In accordance with another aspect, the mating surfaces do not inhibit removal of the valve housing from the storage tray out of the upper tray opening.

In accordance with another aspect, the mating surfaces can be configured to position the valve housing in a specific orientation relative to the storage tray. In one aspect, the valve housing mating surfaces are configured to permit the valve housing to nest within the storage tray in one orientation.

In accordance with another aspect, the mating surfaces are not frictionally engaged with one another.

In accordance with another aspect, the mating surfaces can comprise a male key formed from one of the valve housing and the storage tray and a female key formed from the other of the valve housing and storage tray.

In accordance with another aspect, the female key can be formed as a U-shaped channel in the storage tray. The U-shaped channel can comprise an open end, a closed end, and a channel width (w_{f}) between the open and closed end. In accordance with another aspect, male key can be formed as a protrusion on the valve housing that fits within the U-shaped channel. The protrusion can have a protrusion width (wₚ) that is smaller than the channel width (w_{f}). The term “U-shaped” can include shapes including V-shapes, T-shapes, W-shapes, and other shapes that permit keying.

In accordance with another aspect, the open end of the U-shaped channel can face the upper tray opening such that inverting the storage tray causes the valve housing to fall out of the storage tray when the microbial barrier membrane is removed from the storage tray.

In accordance with another aspect, the storage tray can further comprise one or a plurality of finger holds formed from an external surface of the storage tray. The finger holds can further comprise grip-enhancing ridges.

In accordance with another aspect, the storage tray can comprise inner tray side walls to secure the valve housing. The storage tray can further comprise outer tray side walls that can be spaced sufficiently away from the inner tray side walls to permit a user to grip an upper peripheral edge of the valve housing without touching the outer tray side walls or the tray lip.

In accordance with another aspect, the system can further comprise a moisture-barrier receptacle configured to receive the storage tray and the valve housing retained therein. In one aspect, the moisture-barrier material can also be impermeable to moisture and gas.

In accordance with another aspect, the system can further comprise an outer container configured to receive the moisture-barrier receptacle containing the storage tray and valve housing retained therein. The outer container can comprise one or more sensors, such as a radio frequency identification (RFID) tag, a temperature sensor, or a relative humidity sensor.

In accordance with one aspect, the outer container can comprise an RFID tag and a buffer to reduce signal interference.

In accordance with another aspect, the system can comprise a temperature sensor and a temperature indicator. The temperature indicator can display a signal when the packaging assembly or an enclosed space in a component in the packaging assembly has been subjected to a temperature outside of a predetermined temperature range. In accordance with one aspect, the temperature sensor and the temperature indicator can be provided within the outer container.

In accordance with another aspect, the system can comprise a relative humidity sensor and a relative humidity indicator. The relative humidity indicator can display a signal when a relative humidity in within the packaging assembly or within an enclosed space in a component of the packaging assembly is outside of a predetermined relative humidity range. In accordance with one aspect, the relative humidity sensor and the relative humidity indicator can be provided within the moisture-barrier receptacle.

In accordance with another aspect, the system can further comprise a label with instructions allowing for the aseptic transfer of the bioprosthetic heart valve into the sterile field of the operating room by identifying sterile and non-sterile content.

Each feature or concept outlined above is independent, and can be combined with other features or concepts outlined above or with any other feature or concept disclosed in this application.

In another example, a system for dry storage of a bioprosthetic heart valve is provided. The system can comprise a valve housing dimensioned to removably retain a bioprosthetic heart valve. The valve housing can comprise a cavity dimensioned to accommodate a bioprosthetic heart valve and to retain liquid for hydrating the bioprosthetic heart valve prior to implantation in a patient. The cavity can comprise an upper cavity opening, a bottom wall and an inner side wall contacting the bioprosthetic heart valve when the bioprosthetic heart valve is positioned within the cavity. The inner side wall can inhibit rotational and lateral movement of the bioprosthetic heart valve within the cavity.

A lid can be secured onto the upper cavity opening. The lid can comprise a lid inner surface that faces the bottom wall of the cavity when the lid is in a closed position. The lid can be pivotably movable between an open position and a closed position.

One or more apertures can be provided to permit the passage of sterilizing gas into the cavity when the lid is in the closed position.

In accordance with one aspect, the valve housing is formed entirely by a forming method. The forming method can be one or more selected from the group consisting of: thermoforming, injection molding, blow molding, machining, and 3D printing.

In accordance with one aspect, the forming method is thermoforming.

In accordance with one aspect, the valve housing is made with a material consisting of: a thermoformed plastic. The thermoformed plastic can be polyethylene terephthalate glycol (PETG).

In accordance with one aspect, the valve housing can be formed out of a single, unitary piece of material, as opposed to being constructed by joining separate pieces of material. In one aspect, the valve housing does not comprise separate pieces that are joined together.

In accordance with one aspect, the valve housing can further comprise a cavity lip formed around the upper cavity opening and the lid can be frictionally secured to the cavity lip.

In accordance with one aspect, the valve housing can further comprise first and second offset tabs configured to allow a user to open the lid from the closed position to an open position with a single hand. The first offset tab can be formed from the lid and the second offset tab can be formed from the cavity lip.

In accordance with another aspect, the one or more apertures can be formed as one or more gaps between the lid and the cavity lip. In one aspect, the apertures can be created by a forming method without the need for punching holes or otherwise removing material from the valve housing.

In accordance with another aspect, the lid can comprise a lid periphery with a first set of undulations and the cavity lip can comprise a second set of undulations. The first and second set of undulations can meet to form the one or more apertures when the lid is in a closed position.

In accordance with another aspect, the valve housing can comprise a living hinge between the cavity and the lid.

In accordance with another aspect, the bioprosthetic heart valve can comprise a stent and an external surface. The stent can be made of a metal or metal alloy and the external surface can be a fabric. The fabric can surround at least a portion of the stent and the fabric can have a loft that permits radial compression of the bioprosthetic heart valve. In one aspect, the fabric can surround a portion of the stent. In another aspect, the fabric can surround the entire stent.

In accordance with another aspect, the inner side wall of the cavity can be dimensioned such that opposing surfaces of the inner side wall can compress the bioprosthetic heart valve to removably retain the bioprosthetic heart valve within the cavity.

In accordance with another aspect, the opposing surfaces of the inner side wall can compress only the fabric of the bioprosthetic heart valve and not the stent.

In accordance with another aspect, a diameter (dₕᵥ) of the bioprosthetic heart valve can be substantially the same as a distance (l_{iw}) between opposing surfaces of the inner side wall.

In accordance with another aspect, a distance (l_{iw}) between opposing surfaces of the inner side wall can be less than a diameter (dₕᵥ) of the bioprosthetic heart valve.

In accordance with another aspect, the distance (l_{iw}) between opposing surfaces of the inner side wall can be greater than a diameter (dₛ) of the stent. In accordance with another aspect, the distance (l_{iw}) between opposing surfaces of the inner side wall can be less than a diameter (dₕᵥ) of the bioprosthetic heart valve and greater than a diameter (dₛ) of the stent.

In accordance with another aspect, the lid inner surface can face the bottom wall and can be spaced at a distance that is substantially the same as a length (lₕᵥ) of the bioprosthetic heart valve to inhibit longitudinal movement of the bioprosthetic heart valve within the valve housing when the lid is in the closed position.

In accordance with another aspect, the lid inner surface can comprise a cylindrical lid cavity that can be shaped to accommodate one of an outflow end or an inflow end of the bioprosthetic heart valve, depending on how the bioprosthetic heart valve is oriented in the valve holder. A diameter (d_{lc}) of the cylindrical lid cavity can be larger than the diameter (dₛ) of the stent or the diameter (dₕᵥ) of the bioprosthetic heart valve.

In accordance with another aspect, the valve housing can further comprise indicia identifying the bioprosthetic heart valve. The indicia can be provided on one or both of the lid and the bottom wall. The indicia can indicate a size of the bioprosthetic heart valve. The indicia can be thermoformed on the lid and the bottom wall.

In accordance with another aspect, the bioprosthetic heart valve can be secured within opposing surfaces of the inner side wall by frictional engagement between the inner side wall and fabric.

In accordance with another aspect, the bioprosthetic heart valve is not physically attached, fastened, or snapped into the valve housing.

In accordance with another aspect, the valve housing can further comprise an outer wall. The distance between opposing surfaces of the outer wall can be larger than the distance between the opposing surfaces of the inner side wall. The opposing surfaces of the outer wall do not contact the bioprosthetic heart valve.

Each feature or concept outlined above is independent, and can be combined with other features or concepts outlined above or with any other feature or concept disclosed in this application.

Examples of the packaging system do not include a holder or housing to which the valve is mechanically secured, connected, or attached, for example, using suture, one or more clips, or another mechanical fastener.

Examples of the packing system substantially do not include sterilant in the final product.

Various features as described elsewhere in this disclosure can be included in the examples summarized here and various methods and steps for using the examples and features can be used, including as described elsewhere herein.

### BRIEF DESCRIPTION OF THE DRAWINGS

FIG. 1 is an exploded perspective view of the storage system and includes a microbial-barrier membrane, a bioprosthetic heart valve, a valve housing, and an open storage tray.

FIG. 2A is an exploded perspective view of an example of the open storage tray and the microbial-barrier membrane.

FIG. 2B is a top plan view of the open storage tray of FIG. 2A.

FIG. 2C is a side view of the open storage tray taken from the view indicated in FIG. 2A.

FIG. 2D is a side view of the open storage tray taken from the view indicated in FIG. 2A.

FIG. 3A is a perspective view of an example of the valve housing with the lid in the closed position.

FIG. 3B is a perspective view of the valve housing of FIG. 3A with the lid in the open position.

FIG. 3C is a top view of the valve housing of FIG. 3A with the lid in the open position.

FIG. 3D is a side view of the valve housing of FIG. 3A with the lid in the closed position taken from the view indicated in FIG. 3A.

FIG. 4A is a perspective view of an exemplary bioprosthetic valve.

FIG. 4B is a side view of the bioprosthetic valve of FIG. 4A.

FIG. 5A is a perspective view of the bioprosthetic heart valve of FIG. 4A positioned within the valve housing.

FIG. 5B is a cross-sectional view of valve housing taken along 5B-5B of FIG. 3A.

FIG. 6A is a perspective view of the valve housing of FIG. 3B nested within the storage tray of FIG. 2A.

FIG. 6B is a cross-sectional view of the valve housing and bioprosthetic valve assembly taken along 6B-6B of FIG. 6A.

FIG. 7 is a perspective view of an exemplary moisture-barrier receptacle that can be torn off.

FIG. 8 is a perspective view of an exemplary packaging having sensors, a label, and an indicator.

### DETAILED DESCRIPTION

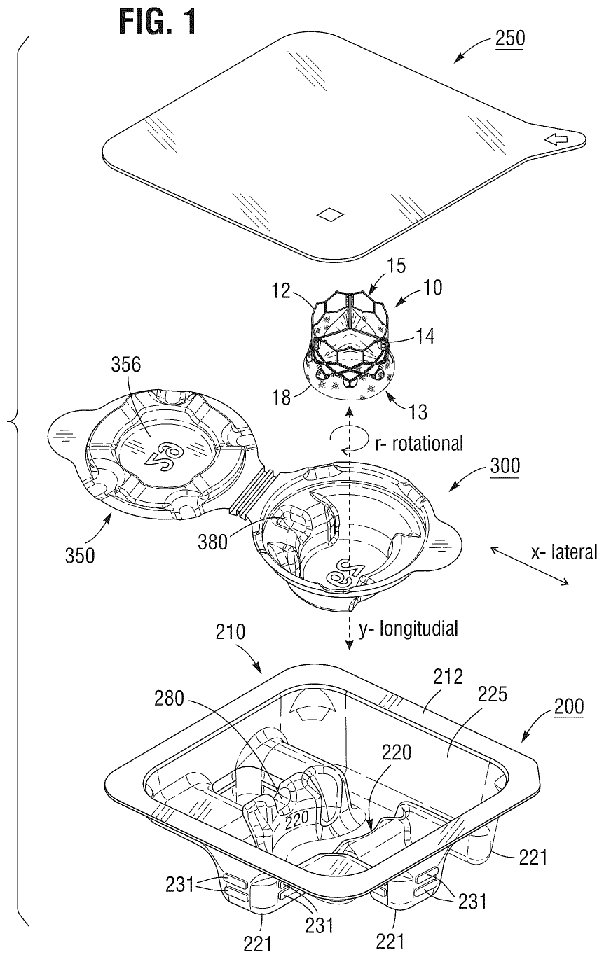

The packaging system (100) is shown in FIG. 1 as generally comprising an open storage tray (200) and a microbial barrier membrane (250) which, when assembled together, provides a fully enclosed cavity that can accommodate a valve housing (300). The valve housing (300) in turn can be dimensioned to retain a bioprosthetic heart valve (10). Each of these components will be described in greater detail with reference to the figures that follow.

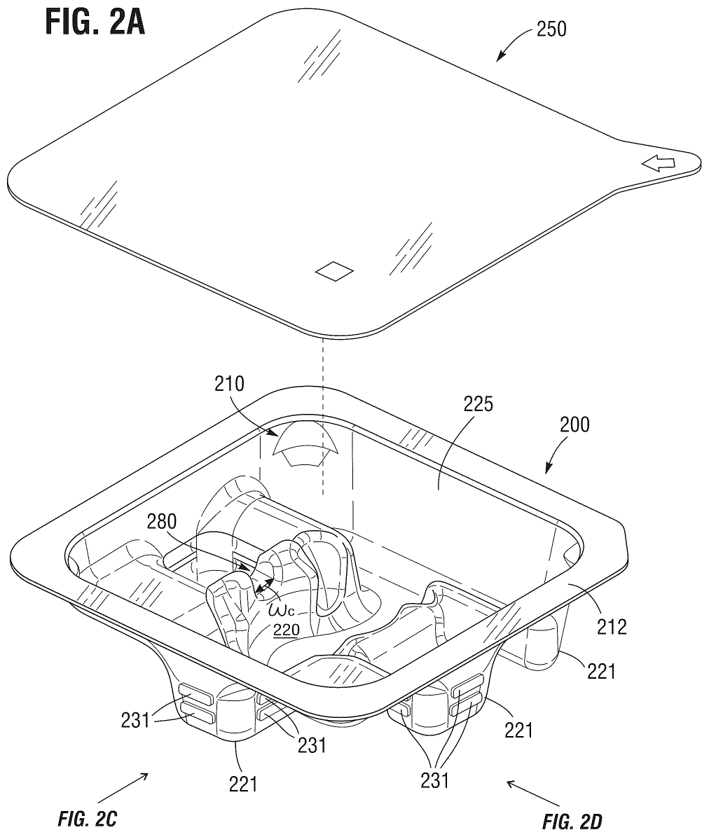

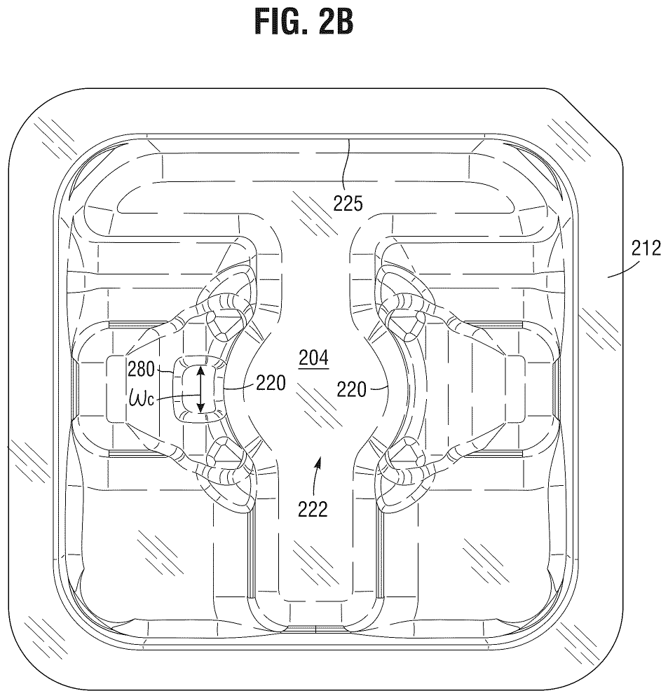

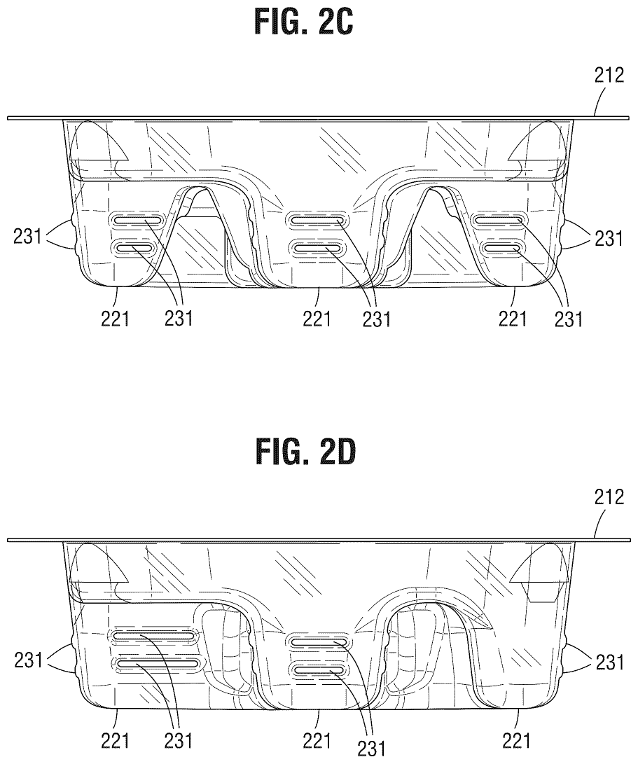

The storage tray (200) is depicted in FIGS. 1 and 2A-2D. The storage tray (200) comprises an open cavity (202) defined by a bottom (204), outer tray side walls (225) and an upper tray opening (210). The upper tray opening (210) terminates at a surrounding tray lip (212). Inner tray side walls (220) are formed within the cavity (202) and define a valve housing retention space therebetween. The inner tray side walls (220) are spaced and configured to house a valve housing (300). A separate microbial barrier membrane (250) can be attached to the tray lip (212) to fully enclose the valve housing (300) inside the cavity (202). The storage tray (200) can additionally feature a grip-enhancing ridges (231) disposed on sides of the formed finger holds (221) to enhance a user's grip of the storage tray (200).

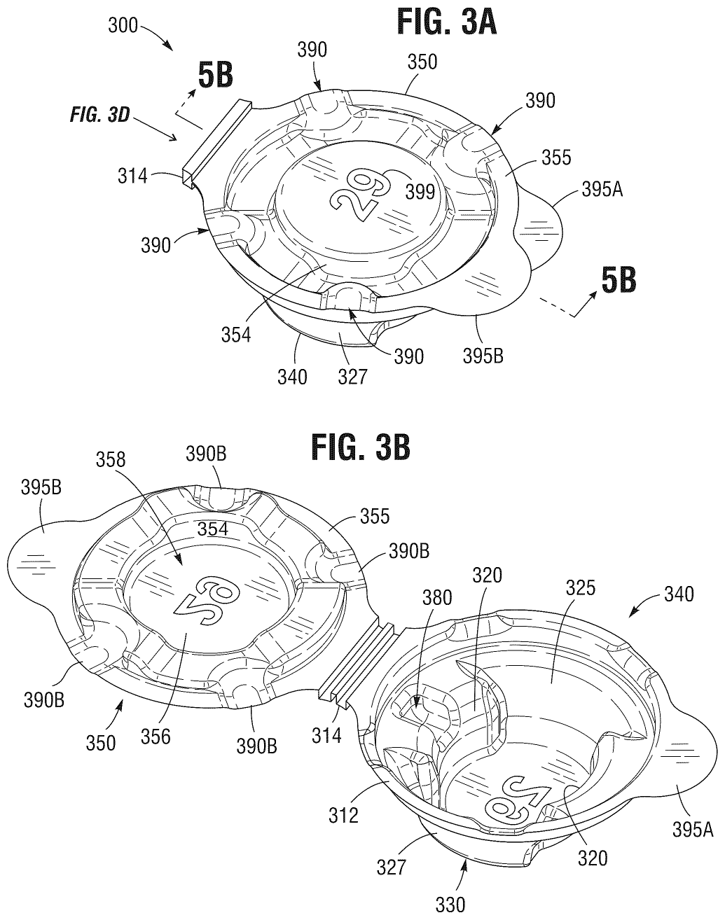

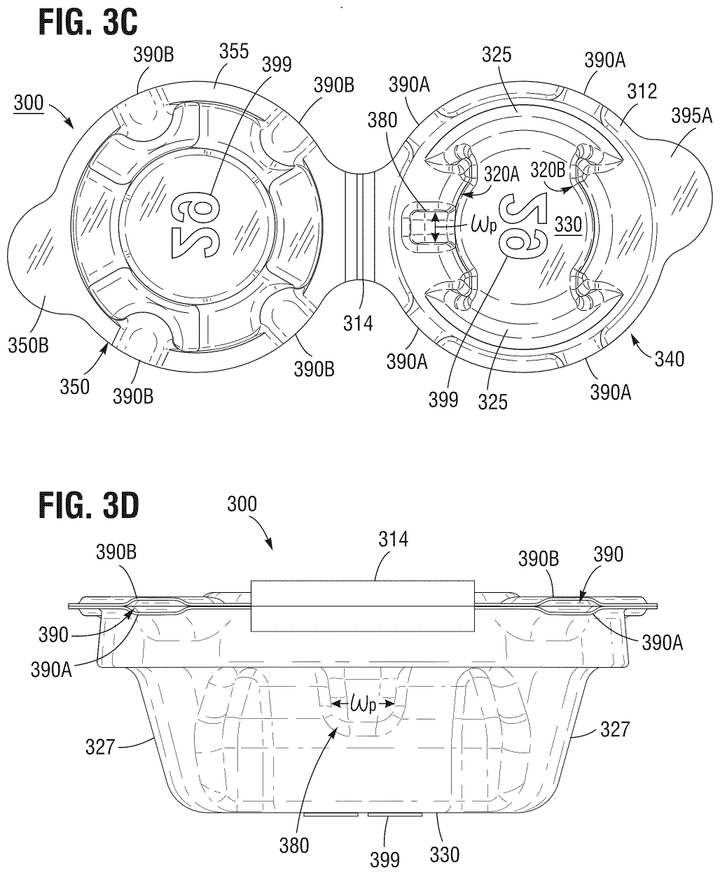

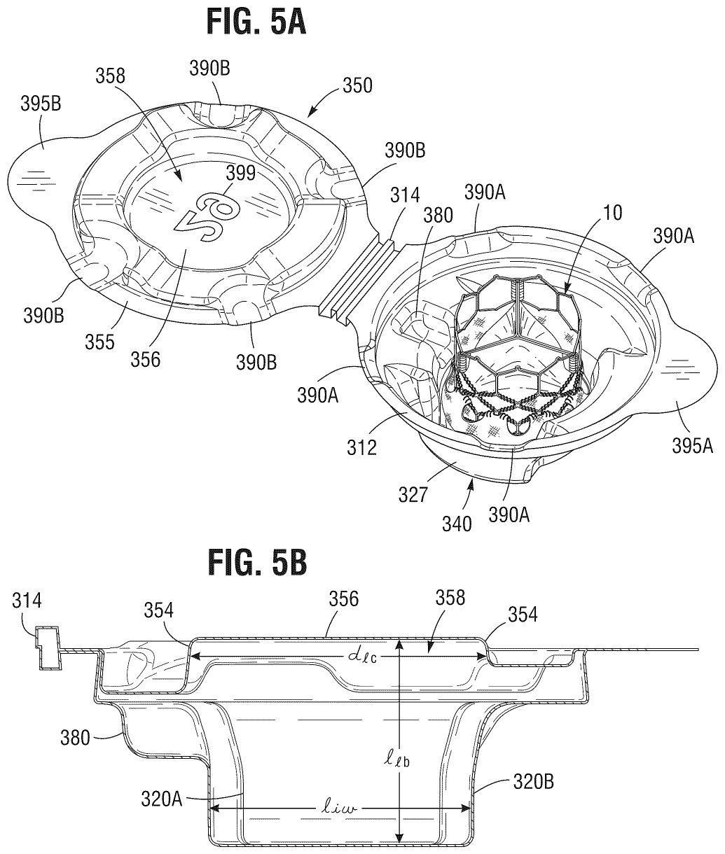

The valve housing (300) is depicted in FIGS. 3A-3D and 5A-5B. The valve housing (300) includes a cavity (340) and a lid (350) that is movable between a closed position (FIG. 3A) and an open position (FIG. 3B). The cavity (340) includes an inner side wall (320) and a bottom wall (330) defining a valve space within which the bioprosthetic heart valve (10) can be retained. An outer side wall (325) can be spaced away from the inner side wall (320) and can provide additional cavity volume to accommodate a hydrating liquid. The additional cavity volume provided by the outer side wall (325) can be in fluid communication with the valve space so that the hydrating liquid can immerse the bioprosthetic heart valve (10).

A lid (350) can be provided to cover an upper cavity opening (310) and enclose the bioprosthetic heart valve (10) within the cavity (340). The lid (350) can be pivotally connected to the cavity (340) by a hinge (360) and can comprise a lid inner surface (356) centrally disposed on the lid (350). The lid (350) can be frictionally secured to the cavity lip (312).

In one example, the hinge (314) is a living or flexible hinge comprising a one or a plurality of bends, as depicted in FIGS. 3B and 5A. Providing a living hinge (314) allows for the molding or forming of the lid (350), hinge (314) and cavity (340) from the same material and into a single integral piece without requiring the joining or physical attachment of separate parts. Other examples of the hinge include a living hinge that does not include any bends. In other examples, the lid and cavity of the valve housing are manufactured as separate components.

A pair of tabs (395A, 395B) may further be provided to allow a user to separate the lid (350) from the cavity (340). In the example depicted in FIGS. 3A-3C, one tab (395A) can be formed from and extend from a cavity lip (312) and the other tab (395B) can be formed from and extend from the lid periphery (355). The tabs (395A, 395B) can be offset from one another to permit the opening of the lid (350) with a single hand. In one method, two fingers may apply opposing pressure to the tabs (395A, 395B) to separate them.

Notably, examples of the packaging system (100) do not include a holder or housing to which the bioprosthetic valve (10) is mechanically secured, connected, or attached, for example, using suture, one or more clips, or another mechanical fastener. Omitting these features can reduce cost by eliminating manufacturing steps, particularly manual steps, and reducing part count, as well as improving the end-user experience by eliminating the step of separating the valve from the holder.

While the packaging system (100) is depicted as housing a bioprosthetic heart valve (10), it is understood that the packaging system (100) can be sized and adapted to house other implantable medical devices requiring dry and sterile storage conditions.

The term “dry” as used herein does not preclude the presence of any water or moisture, including liquid water, within or on a surface of the packaging or bioprosthetic valve. For example, some bioprosthetic valves include tissue in which water is an intrinsic component, in which cases completely eliminating water is not desirable. Additionally, some manufacturing steps can include at least some water, for example, sterilization by ethylene oxide or propylene oxide. Droplets of water may also be present as condensate, particularly at lower temperatures.

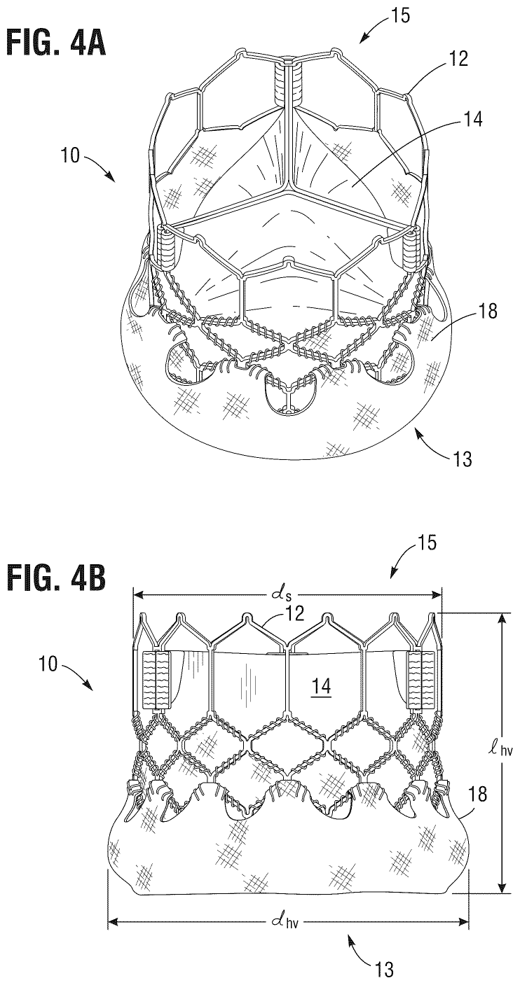

In the exemplary examples, the packaging system (100) can be used to store a bioprosthetic heart valve (10). FIG. 4A-4B depict a bioprosthetic heart valve (10) comprising a stent or frame (12) having a stent diameter (dₛ) and a length (lₕᵥ) between the inflow end (13) and an outflow end (15). A valvular structure (14) is secured to an internal surface of the stent (12) and a fabric (18) surrounds at least a portion of an external surface of the stent (12) at the inflow end (13). The bioprosthetic heart valve (10) can come in a variety of sizes. For a bioprosthetic heart valve (10) that is intended for implantation in a patient's annulus, the bioprosthetic heart valve (10) may come in a variety of diameters (e.g., 20 mm, 23 mm, 26 mm, and 29 mm). Thus, the appropriate bioprosthetic heart valve (10) may be selected based on a measured size of a patient's annulus.

In one example, the fabric (18) can have a thickness, a loft, a nap, or a pile such that the fabric (18) is compressible. A diameter of the heart valve (dₕᵥ) reflects the diameter as measured from the widest portion of the bioprosthetic heart valve that includes the thickness of the fabric. Thus, the diameter of the heart valve (dₕᵥ) will necessarily be greater than the stent diameter (dₛ) taken at the same location of the bioprosthetic heart valve (10). U.S. Pat. No. 11,123,184 is incorporated herein by reference in its entirety as if fully set forth herein. Other examples of the bioprosthetic heart valve have a different structure, for example, one or more of a different stent structure or a different fabric structure.

The components of the packaging system (100) are designed to preserve the integrity of the bioprosthetic heart valve (10) as it is stored and transported. To this end, various features of the valve housing (300) and the storage tray (200) are configured and dimensioned to reduce or inhibit lateral movement (x-axis), longitudinal movement (y-axis) and/or rotational movement (r) about the y-axis of the bioprosthetic heart valve (10) relative to the valve housing (300) and the storage tray (200) while in transit. See FIG. 1.

In one example, opposing surfaces of the inner side wall (320) of the valve housing (300) can be dimensioned and configured to help stabilize the bioprosthetic heart valve (10).

In accordance with this example at least a portion of the opposing surfaces of the inner side wall (320) can be spaced at a distance (l_{iw}) that is approximately equal to or greater than the diameter (dₛ) of the stent (12), but less than the diameter (dₕᵥ) of the heart valve (10): dₕᵥ>l_{iw}≥dₛ

When configured accordingly, the opposing surfaces of the inner side wall (320) can compress the bioprosthetic heart valve (10) and the compressed fabric (18) can provide sufficient friction to inhibit lateral, longitudinal and/or rotational movement of the bioprosthetic heart valve (10) within the valve housing (300).

While having a portion of the opposing surfaces of the inner side wall (320) separated by distance (l_{iw}) allows the bioprosthetic heart valve (10) to be frictionally retained between the opposing surfaces of the inner side wall (320), this frictional retention is not so great that the bioprosthetic heart valve (10) is incapable of or difficultly removable by hand.

In one example, the distance (l_{iw}) is selected such that only the fabric (18) is compressed to reduce the diameter (dₕᵥ) where the compression is applied, but the stent is not compressed such that the diameter (dₛ) of the stent remains unchanged. This frictional retention can be sufficient to inhibit lateral and rotational movement of the bioprosthetic heart valve (10) within the valve housing (300) without permanently distorting the structural components of the bioprosthetic heart valve (10), such as the stent (12). In other words, the frictional retention is tuned to retain the bioprosthetic heart valve (10) without permanently or adversely changing or affecting the structural integrity of the stent (12).

In some examples, a portion of the inner side wall (320) contacting the bioprosthetic heart valve (10) is sufficiently deformable to expand slightly on insertion thereof, thereby frictionally engaging the bioprosthetic heart valve (10) without deforming or otherwise damaging the heart valve. Such a feature is desirable, for example, in cases in which the heart valve (10) includes portions lacking fabric contacting the inner side wall.

It is understood that this frictional retention of the bioprosthetic heart valve (10) provided by the opposing surfaces of the inner side wall (320) can be achieved in a variety of ways. For example, the opposing surfaces of the inner side wall (320) may be separated by the distance (l_{iw}) along its entire height, from the bottom wall (330) towards the cavity opening (310).

Alternatively, only a portion of the opposing surfaces of the inner side wall (320) may be separated by distance (l_{iw}), as may be the case where the distance between a portion of the opposing surfaces of the inner side wall (320) is narrowed.

In a further alternative, the opposing surfaces of the inner side walls may be separated by distance (l_{iw}) in one portion of the inner side walls (320), with the remaining portions being flared to define a distance greater than distance (l_{iw}). Having the opposing surfaces of the inner side wall (320) flared apart towards the cavity opening (310) may facilitate removal of the bioprosthetic heart valve (10).

While the examples depicted in FIGS. 3B-3D depict the inner side wall (320) as having two separate opposing walls (320A, 320B), it is understood that the inner side wall (320) can be configured as a single, continuous wall.

The valve housing (300) can be further dimensioned to inhibit or limit longitudinal movement of the bioprosthetic heart valve (10) within the valve housing (300). In one example, the lid (350) can be configured such that a distance (l_{lb}) between the lid inner surface (356) and the bottom wall (330) is substantially the same as the length (lₕᵥ) of the bioprosthetic heart valve (10) when the lid (350) is in a closed position.

In one example, the lid (350) can further comprise a raised edge (354) extending beyond and surrounding the lid inner surface (356) such that a recessed lid cavity (358) is defined to house one end of the bioprosthetic heart valve (10) (e.g., the inflow end (13) or outflow end (15)).

In one aspect of this example, the distance (l_{lb}) between the lid inner surface (356) and the bottom wall (330) is slightly larger than the length (lₕᵥ) of the bioprosthetic heart valve (10) when the lid (350) is in a closed position.

In one example, the recessed cavity (358) defined by the raised edge (354) houses an end that is not covered by a fabric and can have a diameter (d_{lc}) that is substantially the same as the diameter (dₛ) of the stent (12). In another example, the recessed cavity (358) defined by the raised edge (354) houses an end that is covered by a fabric and thus can have a diameter (d_{lc}) that is substantially the same as the diameter (dₕᵥ) of the bioprosthetic heart valve (10). Having a recessed cavity diameter (d_{lc}) that is slightly larger than the end of the bioprosthetic heart valve (10) housed within the recessed cavity (358) will reduce the likelihood that the raised edge (354) will contact the bioprosthetic heart valve (10) as the lid (350) is pivotally actuated from an open position (FIG. 3B) and a closed position (FIG. 3A). At the same time, the raised edge (354) provides a further structure that can inhibit or limit lateral movement of the bioprosthetic heart valve (10) within the valve housing (300).

As the valve housing (300) provides sterile containment of the bioprosthetic heart valve (10), the valve housing (300) can comprise one or more apertures (390) to permit the passage of a sterilizing gas into the cavity (340) when the lid (350) is in the closed position (FIG. 3A).

In one example, the one or more apertures (390) are formed when the lid (350) is secured to the cavity lip (312). In accordance with the example depicted in FIGS. 3A and 3B, the cavity lip (312) of the valve housing (300) can comprise indentations or undulations (390A) which are curved away from the lid (350) when the lid (350) is in the closed position (FIGS. 3A-3D and 5A). The lid periphery (355) comprises correspondingly located curved indentations or undulations (390B) which curve away from the cavity (340) when the lid (350) is in the closed position (FIG. 3B). When the lid (350) is secured onto the cavity lip (312), the indentations or undulations (390A, 390B) meet to form an aperture or channel (390) through which a sterilizing gas may enter the cavity (340).

In one example, the indentations or undulations (390A, 390B) can be created by thermoforming. In another example, the one or more apertures (390) are not created in the valve housing (300) by punching, cutting or otherwise removing material the valve housing (300), operations which can generate undesired particulates. Thus, in accordance with this example, the valve housing (300) does not comprise any apertures or features that result from the removal of material from the valve housing (300).

Other examples of the valve housing do not include apertures, for example, where a sterilizing gas is not used in the manufacturing process, for example, sterilization by radiation, electron beam, gamma radiation, ultraviolet radiation, microwave radiation, plasma, heat, steam, or liquid sterilant.

While the valve housing (300) is configured to provide secure retention of the bioprosthetic heart valve (10), the valve housing (300) can be shaped and configured to be securely retained within the storage tray (200).

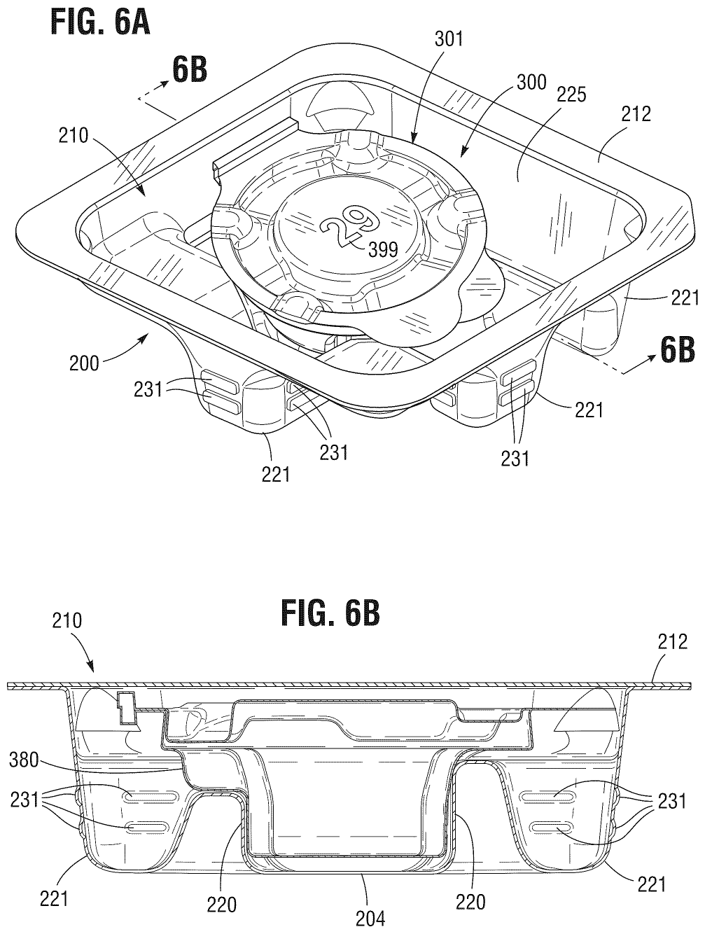

FIGS. 6A-6B depicts the valve housing (300) positioned within the storage tray (200). The inner tray side walls (220) of the storage tray (200) and a facing external surface (327) of the outer side wall (325) of the valve housing (300) are dimensioned, configured and/or shaped to inhibit or limit rotational movement of the valve housing (300) relative to the storage tray (200). This feature is advantageous in providing a further stabilizing feature for the bioprosthetic heart valve (10).

As depicted in FIGS. 6A-6B, the valve housing (300) can be configured to nest entirely within the storage tray (200). In one example, the valve housing (300) can be dimensioned to nest within a valve housing retention space (222) that is provided between opposing surfaces of the inner tray side walls (220). In one aspect, the inner tray side wall (220) partially surrounds the valve housing (300), as depicted in the figures. In another aspect, the inner tray side wall completely surrounds the valve housing (300).

In one example, the valve housing (300) has a curved external wall surface (327). The inner tray side wall (220) can be provided in a correspondingly curved shape to face opposing sides of the external wall surface (327) of the valve housing (300).

It may be desirable for the packaging system (100) to have one or more features that can inhibit or reduce longitudinal, lateral and/or rotational movement of the valve housing (300) relative to the storage tray (200) during transportation.

In one example, the inner tray side walls (220) can be formed to at least partially conform to the contour of the external wall surface (327) to reduce, limit or inhibit lateral movement of the valve housing (300) relative to the storage tray (200). It is understood that a gap may be provided between the inner tray side walls (220) and the external wall surface (327) of the valve housing (300). Notwithstanding this, lateral movement can be inhibited if the gap is sufficiently small. In one example, lateral movement is inhibited so long as the gap between the facing surfaces of the inner tray side walls (220) and the external wall surface (327) is about 5 mm or less, about 4 mm or less, about 3 mm or less, about 2 mm or less, about 1 mm or less, or about 0.5 mm or less or within a range that includes and is between any two of the preceding values. Thus, for example, the gap between the facing surfaces of the inner tray side walls (220) and the external wall surface (327) may be about 3 mm or less.

In another example, a portion of the inner tray side walls (220) and the facing external wall surface (327) can be in physical or frictional contact with one another such that a pulling force (F) is required to disengage the valve housing (300) from the storage tray (200). In one example, the pulling force (F) can be about 10 N or less, about 9 N or less, about 8 N or less, about 7 N or less, about 6 N or less, about 5 N or less, about 4 N or less, about 3 N or less, about 2 N or less, about 1 N or less, or about 0.5 N or less. The pulling force (F) can also be provided within a range that includes and is between any two of the preceding values.

In another example, the inner tray side walls (220) can be spaced away from the facing external wall surface (327) such that there is minimal or no physical contact or frictional engagement between the inner tray side walls (220) and valve housing (300). In accordance with this example, the valve housing (300) can be disengaged or removed from the storage tray (200) by simply inverting the storage tray (200). For example, one can invert the storage tray (200) and to allow the valve housing (300) to simply drop out of upper tray opening (210) and onto a receiving hand or surface in the sterile field. This minimizes the risk of physical contact with the non-sterile parts of the storage tray (200) during the aseptic transfer of the bioprosthetic heart valve (10) to a sterile field. In accordance with this example, no frictional contact is provided between the storage tray (200) and the valve housing (300) and thus, no force is required to remove the valve housing (300) from the storage tray (200).

In one example, longitudinal movement of the valve housing (300) relative to the storage tray (200) can also be further inhibited by relying on certain structural features of the storage tray (200). In one example, the storage tray (200) can be dimensioned such that the microbial barrier membrane (250) is disposed on the tray lip (212) close to or in contact with the lid (350) of the valve housing (300). In accordance with this example, the microbial barrier membrane (250) can be spaced from the top of the valve housing (300) about 5 mm or less, about 4 mm or less, about 3 mm or less, about 2 mm or less, about 1 mm or less, or about 0.5 mm or less when the microbial barrier membrane (250) is attached to the tray lip (212). In accordance with another example, the microbial barrier membrane (250) can be in contact with the lid (350) of the valve housing (300). In another example, the storage tray (200) can include tabs or protrusions that contact or interact with the valve housing (300) to inhibit or limit longitudinal movement of the valve housing when the microbial barrier membrane (250) is assembled on the storage tray (200).

The microbial barrier membrane (250) can be made of a material that can create a microbial barrier. One suitable gas-permeable material is available as a sheet of high-density polyethylene fibers, which is difficult to tear, but can easily be cut, for example, using scissors or a knife. In one example, the gas permeable material permits water vapor and gases can pass through the fibers, but not liquid water. In accordance with this example, nonwoven materials, for example, nonwoven polyethylene fibers (e.g., Tyvek® spunbound polyethylene, DuPont) can be used. Hot-melt adhesives can be used to secure the microbial barrier membrane (250) to the tray lip (212).

The microbial barrier membrane (250) can be adhered to the tray lip (212) with an adhesive. Suitable adhesives can include a hot-melt adhesive, a pressure seal adhesive, or a heat seal adhesive. The microbial barrier membrane (250) can be made from a breathable or gas-permeable material to provide for gas sterilization of the contents sealed within the storage tray (200). One suitable gas-permeable material is a sheet of high-density polyethylene fibers, which is difficult to tear but can easily be cut with scissors. In one example, the microbial barrier membrane, while being permeable to gases, is impermeable to liquid water.

Rotational movement of the valve housing (300) relative to the storage tray (200) can also be inhibited or limited. This can be accomplished by the provision of a mating keyed pair comprising a male key and a female key. In one example, the storage tray (200) can comprise one of the male key or the female key and the valve housing (300) can comprise the other one of the male key or the female key. This keying feature eliminates six degrees of freedom between the valve housing the storage tray (three translational and three rotational), thereby reducing uncertainty that can simplify quality control, facilitate validation, and facilitate regulatory approval, as well as eliminating “rattling” and product shifting that could risk end-user perceptions of poor quality.

In the example illustrated in FIGS. 1, 2A and 2B, 3A-3D, 5B and 6B, the storage tray (200) is depicted as comprising the female key (280) and the valve housing (300) is depicted as comprising the male key (380). It is understood that the assignment of the male or female key to the storage tray (200) or valve housing (300) can be reversed such that the valve housing (300) can comprise the female key (280) and the storage tray (200) can comprise the male key (380). The illustrated example includes one set of keys between the storage tray and valve housing. Other examples can include two or more sets of keys.

In one example, the female key (280) is thermoformed from the storage tray (200) and the male key (380) is thermoformed from the valve housing (300). In one example, the mating keyed pair (280, 380) can be frictionally fitted with close tolerances between the surfaces of the female key (280) and the male key (380). In this example, there is little to no rotational movement of the valve housing (300) relative to the storage tray (200).

In another example, engagement of the mating keyed pair can be configured to not interfere with the removal of the valve housing (300) from the storage tray (200). As previously described, it may be desirable for the valve housing (300) to drop out of the storage tray (200) unassisted when the open end of the storage tray (200) is inverted. Therefore, in one example, the female key (280) may be formed as U-shaped channel, with the opening of the “U” facing the open end of the storage tray (200) and the male key (380) may be formed as a protrusion that fits within the female key (280), preferably without friction.

In a further example, the facing surfaces of the female key (280) and the male key (380) can be fitted such that a small gap exists between the two surfaces. The small gap facilitates removal of the valve housing (300) from the storage tray (200) while inhibiting significant rotational movement of the valve housing (300) within the storage tray (200).

In one example, the gap between the two facing surfaces of the female key (280) and the male key (380) is about 5 mm or less, about 4 mm or less, about 3 mm or less, about 2 mm or about 1 mm or less.

In another example, rotational movement of the valve housing (300) relative to the storage tray (200) is inhibited if the rotational movement of the valve housing (300) about a y-axis (FIG. 1) is 15 degrees or less, 14 degrees or less, 13 degrees or less, 12 degrees or less, 11 degrees or less, 10 degrees or less, 9 degrees or less, 8 degrees or less, 7 degrees or less, 6 degrees or less, 5 degrees or less, 4 degrees or less, 3 degrees or less, 2 degrees or less, 1 degree or less, or within a range that includes and is between any two of the preceding values.

In one example, the storage tray (200) can comprise an outer tray side wall (225) that is spaced away from the inner tray side wall (220). In the example depicted in FIGS. 2A-2D, the outer tray side wall (225) terminates at the tray lip (212) and is spaced sufficiently away from the opposing surfaces of the inner tray side wall (220) to permit a user to grip an upper peripheral edge (301) of the valve housing, as shown in FIGS. 6A-6B.

As previously mentioned, in one example, the tray lip (212) is positioned at a sufficient distance from the inner tray side walls (220) such that a user's finger can grip the valve housing (300) residing in the valve housing retention space (222) without causing the fingers to contact non-sterile parts of the storage tray (200). The non-sterile parts of the storage tray may include the tray lip (212) and the external surface of outer tray side wall (225).

In one example, one or both of the storage tray (200) and the valve housing (300) can be thermoformed entirely of a single material. In one example, the thermoformed material can be a plastic or polymer, such as polypropylene (PP), polystyrene (PS), polyethylene terephthalate (PET), polyethylene terephthalate glycol (PETG), low-density polyethylene (LDPE), high-density polyethylene (HDPE), polyvinyl chloride (PVC), polycarbonate (PC), polymethyl methacrylate (PMMA), acrylonitrile-butadiene-styrene (ABS), as well as blends thereof. In a preferred example, one or both of the storage tray (200) and the valve housing (300) are the same material, for example, PETG. Manufacturing both components from the same material can improve recyclability of the packaging system. The thermoformed material can be completely opaque, semi-opaque, or transparent. In a preferred example, the thermoformed material is transparent to permit visualization of the valve housing (300) contained within the valve housing retention space (222).

In one example, one or both of the storage tray (200) and valve housing (300) can be made of a transparent material. This permits visualization of the bioprosthetic heart valve (10) and the valve housing (300) prior to removing the microbial barrier membrane (250) of the storage tray (200). In one example, identifying indicia (399) may be provided on one or both of the lid (350) and bottom wall (330) of the valve housing (300). Because the storage tray (200) is transparent, the identifying indicia (399) will be visible without removing the microbial barrier membrane (250) of the storage tray (200). As shown in FIGS. 1 and 3A-3D, the identifying indicia can be any information that relates to a feature of the bioprosthetic heart valve (10), such as a size and/or model of the bioprosthetic heart valve (10).

In one example, the packaging system (100) is assembled by placing a bioprosthetic heart valve (10) between opposing surfaces of the inner side wall (320) of the valve housing (300) and closing the lid (350) to enclose the bioprosthetic heart valve (10). The valve housing (300) containing the bioprosthetic heart valve (10) is then placed inside the storage tray (200) such that male key (380) associated with the valve housing (300) is positioned inside the female key (280) associated with the storage tray (200). The microbial barrier membrane (250) is secured to the storage tray (200) using a suitable adhesive and the packaging system (100) to enclose the valve housing (300) and bioprosthetic heart valve (10) and the assembled packaging system is subjected to a sterilization process. The sterilization process is selected based on the characteristics of the medical device and packaging, and may include gas sterilization, radiation, gamma irradiation, electron beam irradiation, microwave irradiation, plasma, ozone, steam, heat. In one example, the sterilization is gas sterilization and, more specifically, sterilization by ethylene oxide. The selected sterilization method preferably does not generate hazardous or toxic by-products.

Examples of the packaging system (100) substantially do not include residual sterilant or a terminal sterilant in the end-user product. The bioprosthetic valve (10) is sterilized using a non-persistent sterilant or sterilizing method, and maintained in a sterile state by the packaging system. Consequently, the end-user is not exposed to residual sterilant or a terminal sterilant when removing the bioprosthetic valve (10) from the packaging system (100), thereby reducing potential exposure to hazardous substances, as well as reducing the use of such substances throughout the supply chain.

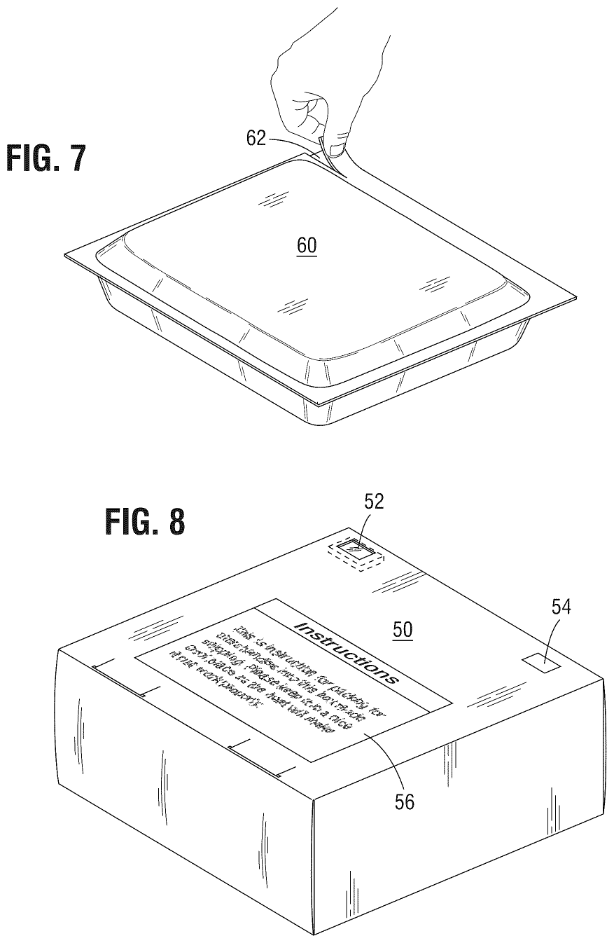

In one example, the assembled packaging system can be further packaged inside a moisture-barrier receptacle (60). The moisture-barrier receptacle (60) can be made of any suitable material that is impermeable to both moisture and also to gas. Examples of such material include a foil pouch, which typically include an aluminum or cobalt foil layer laminated between on or more polymer layers, for example, polyester, polyamide, and/or polyethylene. Some examples include a portion that is configured to be torn off (62), for example, including a notch, tear tab, or tear strip as depicted in FIG. 7. The moisture-barrier receptacle (60) can further be contained in an outer container (50), such as a box or carton.

In one example, the packaging system (100) can comprise one or more sensors (52), such as a radio frequency identification (RFID) tag, including an optional buffer to reduce signal interference, a temperature sensor, and a relative humidity sensor. While the sensor (52) is depicted in FIG. 8 as being disposed within the outer container, it is understood that one or more sensors can be disposed within the moisture-barrier receptacle (60), the storage tray (200) and/or the valve housing (300). In one aspect, one or more sensors (52) can be provided on the outer container (50) and/or within an enclosed space defined within the outer container (50), the moisture-barrier receptacle (60), the storage tray (200) or the valve housing (300). The RFID tag can be used, for example, to uniquely identify a package or device, which is useful in inventorying or reporting the precise identity of the prosthetic valve used in a particular procedure. RFID technology permits automation of steps that are traditionally performed manually, thereby improving efficiency and reducing errors. In some examples, the RFID tag replaces a human-readable identity tag that is often connected to the prosthetic valve, for example, using suture.

In a further example, the packaging system (100) can also comprise one or more indicators (54) that can display a message if the packaging system (100) has been subjected to an environmental condition that exceeds the acceptable limits detected by the one or more sensors for storage of the bioprosthetic heart valve (10). In one aspect, the indicator (54) can be provided on the outer container (50) and/or within an enclosed space defined within the outer container (50), the moisture-barrier receptacle (60), the storage tray (200) or the valve housing (300). In the example depicted in FIG. 8, the indicator (54) can be provided within the outer container (50) but visible through a transparent window without opening the outer container.

In one example, the sensor (52) is a temperature sensor, and the indicator (54) is a temperature indicator that is capable of displaying a signal if, in the course of storage and transit, the outer container (50) has been subjected to a temperature outside of a predetermined and acceptable range for the bioprosthetic heart valve (10).

In another example, the sensor (52) is a relative humidity sensor, and the indicator (54) is a relative humidity indicator. The relative humidity indicator can display a signal the relative humidity sensor detects a relative humidity that is outside of a predetermined and acceptable range for the bioprosthetic heart valve (10).

In one example, instructions for the transfer of the packaging system (100) from a non-sterile to a sterile field can be associated with the outer container (50). The instructions can be provided on a label (56) affixed to the external surface of the outer container (50) as shown in FIG. 8 or provided as a separate sheet included the outer container (50). The instructions may identify the non-sterile components (e.g., the outer container (50) and the moisture-barrier receptacle (60)) and the sterile components (the packaging system 100)).

Once the outer container (50) is received, reference is made to the temperature indicator to ascertain whether the outer container (50) has been subjected to unacceptable temperature. If the outer container (50) has not been subjected to unacceptable temperature excursions, then the moisture-barrier receptacle (60) is removed from the outer container (50). It is understood that these steps are typically carried out in a non-sterile field.

The transfer of the storage tray (200) from a non-sterile field to a sterile field begins with the opening of the moisture-barrier receptacle (60). In one example, the moisture-barrier receptacle (60) comprises a relative humidity sensor. Reference is made to the relative humidity indicator to determine whether the relative humidity has exceeded an acceptable range. In one example, one person in the non-sterile field can open the moisture-barrier receptacle (60) and another person in the sterile field can carefully receive the packaging system (100). In another example, a single person can simply open the moisture-barrier receptacle (60) and simply empty the packaging system (100) onto a surface in the sterile field. It is understood that the person in the non-sterile field avoids any direct physical contact with the packaging system (100). Once in the sterile field, the microbial barrier membrane (250) may be carefully removed from the storage tray (200). The valve housing (300) can then be carefully removed from the storage tray (200) by gripping an upper peripheral edge (301) of the valve housing (300) without touching the outer tray side walls (225) or the tray lip (212).

As previously discussed, the valve housing (300) can be delivered to a sterile area, such as operating room, either by carefully grasping the sides of the valve housing (300) and lifting it from the storage tray (200) or by simply inverting the storage tray (200) to a receiving hand or area and allowing the valve housing (300) to fall out. The valve housing (300) may then be opened by moving the tabs (395A, 395B) apart from one another. In the example depicted in FIGS. 3A-3D, one tab (395A) extends from the cavity lip (312) and the other tab (395B) extends from the lid (350). The tabs (395A, 395B) may be offset to permit separation with a single hand. Once opened, the bioprosthetic heart valve (10) may be hydrated within the cavity (340) by the addition of a liquid solution, such as saline, to soak the valve residing therein prior to implantation.

It should be appreciated that the foregoing description provides an improved packaging assembly featuring an interference force within a desired range over a larger range of interference widths, thereby securely maintaining a bioprosthetic heart valve within the packaging system, while also allowing easy removal of the heart valve from the packaging system without damage or contamination.

Specific methods, devices, and materials are described, although any methods and materials similar or equivalent to those described can be used in the practice or testing of the present examples. Unless defined otherwise, all technical and scientific terms used herein have the same meanings as commonly understood by one of ordinary skill in the art to which these examples belong.

The terms “a,” “an,” and “at least one” encompass one or more of the specified elements. That is, if two of a particular element are present, one of these elements is also present and thus “an” element is present. The terms “a plurality of” and “plural” mean two or more of the specified elements. The term “or” used between the last two of a list of elements means any one or more of the listed elements. For example, the phrase “A, B, or C” means “A, B, and/or C,” which means “A,” “B,” “C,” “A and B,” “A and C,” “B and C,” or “A, B, and C.” The term “coupled” generally means physically coupled or linked and does not exclude the presence of intermediate elements between the coupled items absent specific contrary language.

Without further elaboration, it is believed that the preceding description enables one skilled in the art to make and use the same to the fullest extent. The detailed description provides only the presently preferred examples. Persons skilled in the art will appreciate that various modifications can be made without departing from scope of the disclosure, which is defined only by the following claims.

What is claimed is:

1. A system for dry storage of a bioprosthetic heart valve, the system comprising:
a valve housing dimensioned to removably retain a bioprosthetic heart valve comprising a stent having a stent diameter (dₛ) and a compressible fabric covering at least a portion of an external surface of the stent having a diameter (dₕᵥ), the valve housing comprising:
a cavity dimensioned to accommodate the bioprosthetic heart valve and to receive and retain liquid for hydrating the bioprosthetic heart valve prior to implantation in a patient, the cavity comprising an upper cavity opening, a cavity lip surrounding the upper cavity opening, a bottom wall and an inner side wall;
wherein opposing surfaces of the inner side wall contact the bioprosthetic heart valve when the bioprosthetic heart valve is positioned within the cavity to inhibit rotational and lateral movement of the bioprosthetic heart valve within the cavity;
wherein at least a portion of the opposing surfaces of the inner side wall are spaced to compress only the compressible fabric of the bioprosthetic heart valve and not the stent such that a diameter of the stent (dₛ) remains substantially unchanged;
a lid comprising a lid periphery, the lid being pivotably movable between an open position exposing the upper cavity opening and a closed position to cover the upper cavity opening; and
a plurality of undulations formed on one or both of the lid periphery and the cavity lip;
wherein in the closed position,
at least a portion of the lid periphery and at least a portion of the cavity lip surrounding the upper cavity are joined together;
the plurality of undulations permits the passage of a sterilizing gas between the lid periphery and the cavity lip and into the cavity to sterilize the bioprosthetic heart valve; and
a lid inner surface faces the bottom wall and is spaced at a distance (l_{lb}) that is substantially the same as a length (lₕᵥ) of the bioprosthetic heart valve to inhibit movement of the bioprosthetic heart valve along a longitudinal axis of the bioprosthetic heart valve within the valve housing.

2. The system of claim 1, wherein the valve housing further comprises an outer side wall that is spaced away from the inner side wall, the outer side wall enlarging a volume of the cavity to retain the liquid for hydrating the bioprosthetic heart valve.

3. The system of claim 1, further comprising a storage tray having an upper tray opening and a microbial barrier membrane, the storage tray being configured to receive the valve housing through the upper tray opening and enclose the valve housing when the microbial barrier membrane is secured to a tray lip surrounding the upper tray opening.

4. The system of claim 3, wherein the storage tray is formed from a single, unitary piece of material.

5. The system of claim 3, wherein the storage tray does not comprise separate parts that are physically joined together.

6. The system of claim 3, wherein the valve housing nests entirely within the storage tray and microbial barrier membrane.

7. The system of claim 3, wherein the microbial barrier membrane is permeable to a sterilizing gas.

8. The system of claim 3, wherein the storage tray and the valve housing are shaped to inhibit rotational movement of the valve housing relative to the storage tray when the valve housing enclosed within the storage tray.

9. The system of claim 8, wherein the storage tray and the valve housing each comprise mating surfaces configured to contact one another when the valve housing is placed within the storage tray, wherein the mating surfaces comprise a male key formed from one of the valve housing and the storage tray, and a female key formed from the other of the valve housing and storage tray.

10. The system of claim 9, wherein the female key is formed as a U-shaped channel having an open end, a closed end, and a channel width (w_{f}) between the open and closed ends on the storage tray and wherein the male key is formed as a protrusion on the valve housing that fits within the U-shaped channel, wherein the protrusion has a protrusion width (wₚ) that is smaller than the channel width (w_{f}), further wherein the open end of the U-shaped channel faces the upper tray opening such that inverting the storage tray causes the valve housing to fall out of the storage tray when the microbial barrier membrane is removed.

11. The system of claim 3, further comprising a moisture-barrier receptacle configured to receive the storage tray and the valve housing retained therein, and wherein the moisture-barrier receptacle is impermeable to gas.

12. The system of claim 1 further comprising a label with instructions allowing for aseptic transfer of the bioprosthetic heart valve from the into a sterile field of an operating room by identifying sterile and non-sterile content.

13. A system for dry storage of a bioprosthetic heart valve, the system comprising:
a valve housing dimensioned to removably retain a bioprosthetic heart valve, the valve housing comprising:
a cavity dimensioned to accommodate a bioprosthetic heart valve and to retain liquid for hydrating the bioprosthetic heart valve prior to implantation in a patient, the cavity comprising an upper cavity opening, a bottom wall and an inner side wall contacting the bioprosthetic heart valve when the bioprosthetic heart valve is positioned within the cavity to inhibit rotational and lateral movement of the bioprosthetic heart valve within the cavity;
a lid configured to be secured onto the upper cavity opening, the lid comprising a lid inner surface that faces the bottom wall of the cavity when the lid is in a closed position, the lid being pivotably movable between an open position and a closed position; and
one or more apertures to permit the passage of sterilizing gas into the cavity when the lid is in the closed position.

14. The system of claim 13, wherein the valve housing is made with a material consisting of a polyethylene terephthalate glycol (PETG) thermoformed plastic.

15. The system of claim 14, wherein the valve housing further comprises first and second offset tabs configured to allow a user to open the lid from the closed position to an open position with a single hand.

16. The system of claim 13, wherein the bioprosthetic heart valve includes a fabric, and the bioprosthetic heart valve is secured within opposing surfaces of the inner side wall by frictional engagement between the side wall and the fabric.

17. The system of claim 13, wherein the bioprosthetic heart valve is not physically attached, fastened, or snapped into the valve housing.

18. The system of claim 17, wherein the valve housing further comprises an outer wall, wherein the distance between opposing surfaces of the outer wall (lₒₛ) is larger than the distance (l_{iw}) between the opposing surfaces of the inner side wall and wherein the opposing surfaces of the outer wall do not contact the bioprosthetic heart valve.

19. The system of claim 13, wherein the valve housing is formed from of a single, unitary piece of material.

20. The system of claim 13 further comprising a label with instructions allowing for aseptic transfer of the bioprosthetic heart valve into a sterile field of an operating room by identifying sterile and non-sterile content.
